(19) United States
(12) Patent Application Publication
(10) Pub. No.: US 2014/0275883 A1
(43) Pub. Date: Sep. 18, 2014

(54) WIRELESS SENSORS

(71) Applicant: COVIDIEN LP, Mansfield, MA (US)

(72) Inventors: Charles Haisley, Boulder, CO (US); Sarah Hayman, Boulder, CO (US); Christopher J. Meehan, Golden, CO (US); Daniel Lisogurski, Boulder, CO (US); Andy S. Lin, Boulder, CO (US)

(73) Assignee: COVIDIEN LP, Mansfield, MA (US)

(21) Appl. No.: 13/826,900

(22) Filed: Mar. 14, 2013

(51) Int. Cl. A61B 5/1455; A61B 5/00; A61B 5/1495; A61B 5/0205

(52) U.S. Cl. CPC A61B 5/14552; A61B 5/0205; A61B 5/0004; A61B 5/1495; A61B 5/6806
USPC 600/324

(57) ABSTRACT

Embodiments of the present disclosure relate to medical systems having a wireless medical sensor with a disposable portion and a reusable portion. According to certain embodiments, the disposable portion may include an emitter configured to emit one or more wavelengths of light. The reusable portion may include a power source, such as a battery, for providing power to the emitter and other various components of the sensor. In certain embodiments, the reusable portion may also include a wireless module for communicating with a patient monitor.

### BACKGROUND

The present disclosure relates generally to medical devices and, more particularly, to medical sensors such as those used for pulse oximetry.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. These devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

Pulse oximeters and other types of monitoring devices typically use either disposable sensors, which are discarded after a single use, or reusable sensors. Disposable sensors may provide convenience in the medical setting and may lower the risk of transferring bacteria or disease between patients. However, certain sensor components, especially electrical components, within the sensor may be expensive. Reusable sensors may lower the overall cost of the sensor per use, but these sensors generally require more effort and time on the part of the medical practitioner. For example, such reusable sensors must be thoroughly disinfected after each use.

Additionally, typical pulse oximetry sensors may communicate with a patient monitor using a communication cable. For example, a sensor may use such a communication cable to send a signal corresponding to a measurement performed by the sensor to the patient monitor for processing. However, the use of communication cables may limit the applications available, as the cables may become prohibitively expensive at long distances as well as limit a patient's range of motion by physically tethering the patient to a monitoring device. Although wireless sensors may transmit information without the need for a communication cable, the sensors typically rely on wireless transceivers for communication and batteries to power the sensor. Such wireless transceivers and batteries are often relatively expensive components of a sensor, and thus are generally impractical for use in disposable sensors. Additionally, certain features, such as the wireless transceiver and batteries, may be bulky. For example, in such wireless sensors, a large portion of the bulk and weight of the sensor may be attributable to the battery used to power the sensor, and thus, it may be difficult to adequately and comfortably secure the sensor to the patient.

### BRIEF DESCRIPTION OF THE DRAWINGS

Advantages of the disclosed techniques may become apparent upon reading the following detailed description and upon reference to the drawings in which:

FIG. 1 is a front perspective view of a monitoring system, in accordance with an embodiment;

FIG. 2 is a top view of a sensor having a disposable portion and a reusable portion coupled via a connector in accordance with an embodiment;

FIG. 3 is a top view of the sensor having the disposable portion and the reusable portion coupled via a flex circuit in accordance with an embodiment;

FIG. 4 is a top view of the sensor having the disposable portion and the reusable portion disposed on a hand of a patient in accordance with an embodiment;

FIG. 5 is a top view of the sensor having the disposable portion and the reusable portion coupled to a glove in accordance with an embodiment;

FIG. 6 is a top view of the sensor having the disposable portion and the reusable portion, wherein the reusable portion includes a detector, in accordance with an embodiment;

FIG. 7 is a side view of the reusable portion of the sensor of FIG. 6, in accordance with an embodiment;

FIG. 8 is a bottom view of the reusable portion of the sensor of FIG. 7 in accordance with an embodiment;

FIG. 9 is a front perspective view of the sensor of FIG. 6 having the disposable portion and the reusable portion coupled to a headband in accordance with an embodiment;

FIG. 10 is a side view of the sensor of FIG. 6 coupled to the headband of FIG. 9;

FIG. 11 is a side view of the sensor of FIG. 6 within a pocket of the headband;

FIG. 12 is a top view of the sensor having the disposable portion and the reusable portion, wherein the disposable portion has an elongated sensor body, in accordance with an embodiment;

FIG. 13 is a front perspective view of a wrist or ankle band having a first window for the disposable portion and a second window for the reusable portion in accordance with an embodiment;

FIG. 14 is a front view of a wrap having a first window for the disposable portion and a second window for the reusable portion in accordance with an embodiment; and

FIG. 15 is a side view of the wrap of FIG. 14 aligned with the sensor having the disposable portion and the reusable portion in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In certain circumstances, it may be desirable to have a medical sensor having both a disposable portion and a reusable portion that function together for patient monitoring. As discussed above, disposable sensors may provide convenience in the medical setting and may lower the risk of transferring bacteria or disease between patients, while reusable sensors may generally lower the overall cost per sensor. Thus, a sensor having both the disposable portion and the reusable portion may combine the convenience and other benefits of disposable sensors with a reduced cost per sensor provided by the reusable portion.

Additionally, it may be desirable to provide a wireless sensor having both the disposable portion and the reusable portion, as wireless sensors may allow for increased mobility of the patient as compared with traditional wired sensors. However, as noted above, such wireless sensors require a wireless transceiver to communicate with a monitor and a power source (e.g., a battery) to power the various components within the sensor. The wireless transceiver and/or battery may be relatively expensive components, and thus may not be well-suited for use in disposable sensors. Therefore, the present embodiments are generally directed to a medical sensor having both a disposable portion and a reusable portion that function together for patient monitoring, and the reusable portion may generally include certain components that are easily reused or relatively expensive sensor components (e.g., the wireless transceiver) to reduce the cost per sensor.

Furthermore, as described above, certain components of wireless sensors (e.g., the wireless transceiver and/or the battery) may be relatively large and bulky. Accordingly, it may be desirable to place these components in more comfortable locations on the patient and/or to use various wraps and bands to securely and comfortably couple the sensor, to the patient.

Monitoring systems and wireless sensors in accordance with the present disclosure may provide certain advantages over typical wired disposable or reusable sensors. For example, such wireless sensors do not require cables to communicate with a monitor, which may reduce interference from such cables and also allow for increased mobility of a patient. Additionally, having both a disposable portion and a reusable portion may provide significant cost savings over traditional disposable sensors. For example, having both the disposable portion and the reusable portion enables certain components to be discarded, while other components may be reused. Generally, less expensive components (such as an emitter) or smaller components may be disposed in the disposable portion, which allows easy application of such components to the patient. However, certain relatively expensive components (such as wireless transceivers, batteries, and/or detectors) or components that may be readily reused may be disposed in the reusable portion, thus reducing the cost per sensor. In some cases, separation of components into the disposable portion and the reusable portion may also enable the system to include larger components, such as a larger, long-lasting battery and/or a more powerful wireless transceiver. Additionally, separation of components into the disposable portion and the reusable portion may enable large components (e.g., a large battery) to be positioned away from the measurement site in a suitable or more comfortable location (e.g., the back of a hand of the patient) during a monitoring session.

As discussed in detail below, the disposable portion and the reusable portion may be coupled together in any suitable way, and the various components of the sensor (e.g., emitter, detector, wireless transceiver, battery, calibration element, etc.) may be arranged in any suitable manner within the disposable portion and the reusable portion. For example, in some embodiments, as described in more detail below, the disposable portion may include the emitter, the detector, and the calibration element, while the reusable portion may include the battery and the wireless transceiver. In certain embodiments, the disposable portion may include the emitter and the calibration element, while the reusable portion may include the detector, the battery, and the wireless transceiver, for example. In other embodiments, the disposable portion may include the emitter, detector, calibration element, and battery, while the reusable portion may include the wireless transceiver, for example.

Additionally, the disposable portion and the reusable portion of the wireless sensors described herein may be configured to be positioned on a variety of tissue locations on a patient, such as on a finger, a toe, a foot, an ankle, an ear, a wrist, a forehead, or any other appropriate measurement site. As discussed in more detail below, such sensors may be utilized for adults and neonates alike, and the various portions of the sensors may be applied to the patient through various means, including adhesives, garments, wraps, and so forth. For example, in some embodiments, the sensor may be fitted into or placed against a wearable garment, such as a glove or headband.

Although the embodiments described below generally relate to wireless photoplethysmography, disclosed embodiments may be adapted or configured to obtain a variety of medical measurements with a suitable medical sensor. For example, the system and devices described herein may, additionally or alternatively, be configured to measure patient temperature, transvascular fluid exchange volumes, tissue hydration, blood flow, blood microcirculation, respiration, ECG, non-invasive blood pressures (NIBP), blood pulse transit time, and/or others.

With the foregoing in mind, FIG. 1 depicts an embodiment of a wireless medical monitoring system 10 that includes a sensor 12 having both a disposable portion 14 and a reusable portion 16. Although only one disposable portion 14 and one reusable portion 16 are shown, it is contemplated that the system 10 may include a plurality of disposable portions 14 and/or a plurality of reusable portions 16. The disposable portion 14 and the reusable portion 16 may be coupled together in any suitable manner, such as via a flex circuit 18, a cable, or a wire, for example. As shown, the system 10 may include a patient monitor 20 that communicates wirelessly with the sensor 12.

The patient monitor 20 may include a display 22, a wireless module 24 for transmitting and receiving wireless data, a memory, a processor, and various monitoring and control features. Based on data received from the wireless sensor 12, the patient monitor 20 may display patient measurements and perform various measurement or processing algorithms. For example, when the system 10 is configured for pulse oximetry, the patient monitor 20 may perform blood oxygen saturation calculations, pulse measurements, and other measurements based on the data received from the wireless sensor 12. Furthermore, to provide additional functions, the patient monitor 20 may be coupled to a multi-parameter patient monitor 26, for example, via a cable 28 connected to a sensor input port or via a cable 30 connected to a digital communication port. The multi-parameter module 26 may process and/or display physiological parameters from other sensors in addition to the data from the monitor 20 and sensor 12.

Like the patient monitor 20, the sensor 12 may include a wireless module 32. The wireless module 32 of the wireless sensor 12 may establish a wireless communication 34 with the wireless module 24 of the patient monitor 20 using any suitable protocol. By way of example, the wireless modules 24, 32 may be capable of communicating using the IEEE 802.15.4 standard, and may communicate, for example, using ZigBee, WirelessHART, or MiWi protocols. Additionally or alternatively, the wireless modules 24, 32 may be capable of communicating using the Bluetooth standard or one or more of the IEEE 802.11 standards. In an embodiment, the wireless module 32 may include a transmitter (such as an antenna) for transmitting wireless data, and the wireless module 24 includes a receiver (such as an antenna) for receiving wireless data. In an embodiment, the wireless module 32 also includes a receiver for receiving instructions (such as instructions to switch modes), and the wireless module 24 also includes a transmitter for sending instructions to the sensor 12.

FIG. 2 depicts an embodiment of the wireless sensor 12, including the disposable portion 14 and the reusable portion 16. As illustrated, the disposable portion 14 may have a sensor body 40, which may support one or more optical components, such as one or more emitters 42 configured to emit light at certain wavelengths through a tissue of the patient and/or one or more detectors 44 configured to detect the light after it is transmitted through and/or absorbed by the blood and/or tissue of the patient. Additionally, the sensor body 40 of the disposable portion 14 may house other components, such as a calibration element 46 configured to provide information related to the emitter 42 and/or the detector 44, for example. Although not shown in FIG. 2, it should be understood that the sensor body 40 may house other components, such as a battery, for example. The sensor body 40 may be formed from any suitable material, including rigid or conformable materials, such as foam or other padding materials (e.g., a sponge or gel), fiber, fabric, paper, rubber or elastomeric compositions (including acrylic elastomers, polyimide, silicones, silicone rubber, celluloid, PMDS elastomer, polyurethane, polypropylene, polyethylene, acrylics, nitrile, PVC films, acetates, and latex). The disposable portion 14 may also take any suitable form to facilitate patient monitoring. In some embodiments, the disposable portion 14 may be configured to clip to or to wrap around the tissue of the patient, for example. In certain embodiments, the disposable portion 14 may have an adhesive surface to adhere the disposable portion 14 to the patient's skin or to a mounting surface of a band or a wrap, as described in more detail below. As noted above, the disposable portion 14 may be configured to be positioned on a variety of tissue locations on a patient, such as on a finger, a toe, a foot, an ankle, a wrist, or a forehead.

The emitter 42 and detector 44 may be generally configured for patient monitoring and may be arranged in a reflectance or transmission-type configuration with respect to one another. For example, in some embodiments in which the sensor 12 is configured for use on a patient's finger, the emitter 42 and detector 44 may be in a reflectance or a transmission configuration. In embodiments in which the sensor 12 is configured for use on a patient's forehead, the emitter 42 and detector 44 may be in a reflectance configuration. Regardless of the particular arrangement of the emitter 42 and detector 44, the emitter 42 may be a light emitting diode, a superluminescent light emitting diode, a laser diode or a vertical cavity surface emitting laser (VCSEL). Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. In certain embodiments, the sensor 12 may be configured to perform traditional pulse oximetry measurements, regional pulse oximetry measurements, or the like. In embodiments where the sensor 12 is configured to perform regional pulse oximetry measurements, the emitter 42 may include two or more LEDs, each LED being configured to emit a different wavelength of light.

The emitter 42 may be configured to emit at least two wavelengths of light, e.g., red and infrared (IR) light, into the tissue of the patient. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. Regardless of the number of emitters 42 or the number of LEDs within each emitter 42, light from the emitter 42 may be used to measure, for example, oxygen saturation, water fractions, hematocrit, or other physiologic parameters of the patient. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

The detector 44 may be an array of detector elements that may be capable of detecting light at various intensities and wavelengths. In one embodiment, light enters the detector 44 after passing through the tissue of the patient. In another embodiment, light emitted from the emitter 42 may be reflected by elements in the patent's tissue to enter the detector 44. The detector 44 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by the detector 44, and when more light at a certain wavelength is transmitted, more light of that wavelength is typically received from the tissue by the detector 44. After converting the received light to an electrical signal, the detector 44 may send the signal to the monitor 20, where physiological characteristics may be calculated based at least in part on the absorption and/or reflection of light by the tissue of the patient. In embodiments where the sensor 12 is configured for regional saturation monitoring, two detectors 44 may be provided, with one detector 44 relatively close (e.g., proximal) to the emitter 42 and one detector 44 relatively far (e.g., distal) from the emitter 42.

As noted above, in certain embodiments, the disposable portion 14 of the sensor 12 may also include a calibration element 46 that may provide signals indicative of the wavelength of one or more light sources of the emitter 42, which may allow for selection of appropriate calibration coefficients for calculating a physical parameter such as blood oxygen saturation. The calibration element 46 may, for instance, be a coded resistor, EPROM or other coding devices (such as a capacitor, inductor, PROM, RFID, parallel resident currents, barcode, or a colorimetric indicator) that may provide a signal to the monitor 20. The signals may be related to the characteristics of the sensor 12 to enable the microprocessor to determine the appropriate calibration characteristics of the sensor 12, for example.

As described above, the sensor 12 may have both the disposable portion 14 and the reusable portion 16, and the disposable portion 14 may be coupled to the reusable portion 16 through any suitable means. In certain embodiments, as shown in FIG. 2, the disposable portion 14 and the reusable portion 16 may be coupled by a connector 48. For example, a first end 50 of the connector 48 may be attached to or extend from a disposable portion 14, while a second end 52 of the connector 48 may be attached to or extend from the reusable portion 16. The first and second ends 50, 52 may be complementary and may be configured to be coupled together to form an interlocking connection. For example, the first end 50 may have slots 54 which correspond to (e.g., receive, connect with) pins 56 disposed within the second end 52, or vice versa. In some embodiments, when the portions 14, 16 are coupled together by the connector 48, the sensor body 40 of the disposable portion 14 and a sensor body 58 of the reusable portion 16 are not in direct contact with one another and/or do not overlap. Regardless of the form of the connector 48, the connector 48 may generally be configured to electrically and physically couple the disposable portion 14 and the reusable portion 16.

The reusable portion 16 may include any of a variety of components to facilitate patient monitoring, and the components may be arranged within the reusable portion 16 in any suitable manner. In the depicted embodiment, the reusable portion 16 includes the sensor body 58 which may support certain electrical components and/or power sources for the sensor 12. For example, the sensor body 58 of the reusable portion 16 may support the wireless module 32 for wirelessly communicating with the monitor 12. Additionally, the reusable portion 16 may support a battery 60 configured to supply power to various components (e.g., the emitter 42) of the sensor 12. The reusable portion 16 may also include circuitry 62 (e.g., a low power circuit board) to control the operation of various components (e.g., the emitter 42, the detector 44, etc.) of the sensor 12. In some embodiments, the reusable portion 16 may include a battery meter that may provide a visible indication of battery life, as described in more detail below. Thus, in the sensor 12 illustrated in FIG. 2, the reusable portion 16 generally forms a reusable battery pack, which may be connected to and may supply power to the disposable portion 14 via the connector 48. Additionally, in operation, the reusable portion 16 may be easily replaced without disturbing (e.g., removing) the disposable portion 14 on the tissue of a patient, thus enabling replacement of the battery 60 and providing a way to power the disposable portion 14 over relatively long periods of time (i.e., with multiple different reusable battery packs). As discussed above, it may be desirable to provide certain components, such as the wireless module 32 and/or the battery 60, within the reusable portion 16 of the sensor 12 for cost savings.

The sensor body 58 of the reusable portion 16 may be formed from any suitable material, including rigid or conformable materials, such as foam or other padding materials (e.g., a sponge or gel), fiber, fabric, paper, rubber or elastomeric compositions (including acrylic elastomers, polyimide, silicones, silicone rubber, celluloid, PMDS elastomer, polyurethane, polypropylene, polyethylene, acrylics, nitrile, PVC films, acetates, and latex). In some embodiments, the body 58 of the reusable portion 16 may be a water-proof housing and may encapsulate the battery 60 and the various electrical components disposed therein. Such a configuration may protect such components, and may also allow the reusable portion 16 to be easily cleaned and thoroughly disinfected after each use. For example, the body 58 may enable the reusable portion 16 to be submerged in a disinfecting solution without damaging the components within the body 58.

The reusable portion 16 may generally take any form that enables the reusable portion 16 to be coupled to the disposable portion 14 and to be comfortably positioned on the patient. In particular, the reusable portion 16 may be configured to be positioned on a variety of tissue locations on a patient, such as on a finger, a toe, a foot, an ankle, a wrist, or a forehead. In some embodiments, the reusable portion 16 may be configured to clip to or to wrap around the tissue of the patient, for example. In certain embodiments, the reusable portion 16 may have an adhesive surface to adhere to the patient or to another surface, such as a mounting surface of a band or a wrap, as described in more detail below. In particular, as shown in FIG. 2, the reusable portion 16 may have one or more extensions 64 (e.g., wings, arms, etc.) that may be configured to wrap around a digit of the patient. The extensions 64 may be configured to attach to each other so as to securely wrap around the tissue of the patient. In certain embodiments, the extensions 64 may have a hook and loop mechanism that enables the reusable portion 16 to be easily fastened around the tissue of the patient and easily removed for reuse. In other words, a first extension 64a may include a hook material while a second extension 64b may include a loop material (or vice versa), thus enabling the first extension 64a and the second extension 64b to attach to each other as the extensions 64 wrap around the tissue of the patient. In certain embodiments, the extensions 64 may provide a relatively large adhesive patient contacting surface to couple the reusable portion 16 to the tissue of the patient, such as shown and discussed below with respect to FIG. 4.

In some embodiments, a disposable cover may be provided to cover (e.g., surround, fit around, etc.) at least part of the reusable portion 16. Thus, the disposable cover may fit or wrap around the body 58 of the reusable portion 16. For example, the disposable cover may include hook and loop material to secure the disposable cover to or around the reusable portion 16. The cover may be wrapped around the reusable portion 16 before the reusable portion 16 is coupled to the disposable portion 14 for patient monitoring. In some embodiments, the disposable cover may be attached to and may extend from the disposable portion 14, thus forming a platform, pocket, or cavity for receiving and covering the reusable portion 16 when the reusable portion 16 is coupled to the disposable portion 14. The portion of the disposable cover that extends from the disposable portion 14 may have an adhesive surface for receiving and holding the reusable portion 16 and/or may include a flap that can be wrapped around the reusable portion 16. Regardless of the form, the disposable cover may generally be configured to separate the reusable portion 16 from the tissue of the patient and/or the ambient environment in order to protect the reusable portion 16 and/or to limit the spread of bacteria or disease as the reusable portion 16 is reused or transferred from patient to patient. At the conclusion of the patient monitoring session, the disposable cover may be easily removed and discarded, and the reusable portion 16 may be retained for reuse.

Additionally, each of the portions 14, 16 of the sensor 12 may be configured to be placed on or proximate to (e.g., near, adjacent, etc.) the patient's skin for patient monitoring. In some embodiments, the portions 14, 16 of the sensor 12 may be configured to attach to or be coupled to the patient. For example, the sensor body 40, 58 may include an adhesive or other gripping surface configured to secure the sensor 12 to the patient's skin or to another suitable surface, such as a mounting surface of a band or a wrap, as described below. As noted above, in some circumstances, the disposable portion 14 may remain on the patient for long periods of time. Therefore, it may be desirable to be able to easily replace the reusable portion 16, to provide power (e.g., via battery 60) to the disposable portion 14 over a long period of time, for example. In such cases, the reusable portion 16 may be detached from the disposable portion 14 (or from the flex circuit 18). A new (e.g., charged) reusable portion 16 may then be coupled to the disposable portion 14, without having to remove the disposable portion 14 from the patient. In other cases, the disposable portion 14 may become dislodged or may require replacement, or a different type of disposable portion 14 (e.g., a disposable portion 14 having a different type or configuration of emitters 42) may be desired. In such cases, the disposable portion 14 may be disconnected from the reusable portion 16. Subsequently, the new disposable portion 14 can be easily coupled to the reusable portion 16, without having to remove the reusable portion 16 from the patient.

FIG. 3 illustrates another embodiment of the wireless sensor 12 including the disposable portion 14 and the reusable portion 16. Each of the portions 14, 16 may have similar components (e.g., emitter 42, detector 44, calibration element 46, battery 60, etc.) as discussed above with respect to FIG. 2; however, the portions 14, 16 are coupled by the flex circuit 18 rather than the connector 48. The flex circuit 18 may electrically and physically couple the portions 14, 16, and may allow the reusable portion 16 (e.g., the reusable battery pack) to power the electrical components of the disposable portion 14. The flex circuit 18 may be desirable in certain circumstances, as the flex circuit 18 may generally enable customized placement of the portions 14, 16 for various patients and more relative movement between the portions 14, 16 as compared with the connector 48 of FIG. 2. Furthermore, in some embodiments, the flex circuit 18 may be integrated or attached to either the disposable portion 14 or the reusable portion 16. For example, flex circuit 18 may be integrated into the disposable portion 14, and can couple the disposable portion 14 to the reusable portion 16 for patient monitoring. At the conclusion of the monitoring session, the disposable portion 14 and its integrated or attached flex circuit 18 may be discarded.

Additionally, in the embodiment depicted in FIG. 3, the reusable portion 16 also has a different form and configuration than the reusable portion 16 of FIG. 2. As shown, the reusable portion 16 has an annular (e.g., ring) shape and is configured to fit (e.g., slide) over an appendage, such as a finger or a wrist, of the patient. The reusable portion 16 may be adjustable in size (e.g., circumference) in order to adapt to the different anatomies of various patients. Furthermore, the reusable portion 16 may be made from any suitable flexible material, such as silicone.

In some circumstances, the reusable portion 16 may be too bulky or large for placement over (or adjacent to) certain measurement sites, such as a fingertip of the patient, for example. In other words, it may uncomfortable or impractical to position the reusable portion 16 adjacent to the disposable portion 14 or on certain body locations. Additionally, in some systems, it may be desirable for the reusable portion 16 to include a relatively large battery 60 for long-lasting power and/or relatively large circuitry 62 for higher processing capabilities. In such cases, the reusable portion 16 may be configured to be disposed away from the measurement site and/or at a different body location from the disposable portion 14. For example, the reusable portion 16 may be configured to be disposed on a hand of the patient when the disposable portion 14 is disposed on the finger of the patient. FIG. 4 illustrates one embodiment of the sensor 12 having the reusable portion 16 disposed on a back side (e.g., dorsal side) of the hand of the patient. The reusable portion 16 may be coupled to the disposable portion 14 via a cable or the flex circuit 18 of a suitable length. As noted above, in such configurations, even a relatively large reusable portion 16 may be comfortably placed on the patient. Thus, the reusable portion 16 may desirably accommodate a larger, longer-lasting battery 60 to power the electrical components (e.g., emitter 42, etc.) of the sensor 12. Additionally, the reusable portion 16 may be large enough to accommodate relatively powerful processing circuitry within the circuit board 62. Thus, in some embodiments, the reusable portion 16 may be configured to process physiological data and to calculate physiological parameters, for example.

In some embodiments, a garment 70 (e.g., a band, glove, sock, etc.) may be provided to protect and/or to secure the disposable portion 14 and/or the reusable portion 16 to the tissue of the patient. The garment 70 may provide additional benefits, such as regulating (e.g., warming) the temperature of the tissue of the patient and lowering the incidence of vasoconstriction. The garment 70 may be particularly useful for patients having low perfusion. For example, as shown in FIG. 5, the garment may be a glove 70 that is configured to be placed on the hand of the patient. The glove 70 may comprise any suitable material, including an elastic material. The glove 70 may be disposable or reusable. In some embodiments, one or more of the portions 14, 16 of the sensor 12 may first be adhered to the hand of the patient, and the glove 70 may be applied over one or more of the portions 14, 16. In such cases, the glove 70 may exert a normal force against one or more of the portions 14, 16 to press one or more of the portions 14, 16 against the skin of the patient. In some embodiments, one or more of the portions 14, 16 may not have the patient-contacting adhesive surface, and the glove 70 may be utilized to provide a sufficient securing force such that one or more of the portions 14, 16 (e.g., the portion 14, 16 covered by the glove 70) is securely coupled to the hand of the patient without the use of adhesive. In some embodiments, the glove 70 may include a gripping material with a relatively high coefficient of friction on a patient-facing surface of the glove 70 to reduce movement of the glove 70 relative to the patient during patient monitoring.

The glove 70 may alternatively provide a mounting surface or element (not shown) or a pocket 72 configured to receive and to hold the reusable portion 16 of the sensor 12, for example. In some embodiments, the mounting element provides an adhesive surface or a mechanical attachment (e.g., a snap, a clip, etc.) for mounting the reusable portion 16 on an exterior surface of the glove 70. In some embodiments, as shown in FIG. 5, the glove 70 may have pocket 72 configured to surround the reusable portion 16. The pocket 72 may be positioned generally centrally on a region of the glove 70 that is adjacent to the back side (e.g., dorsal side) of the patient's hand. In operation, the glove 70 may be applied to the patient, and the reusable portion 16 may be inserted or placed into the pocket 72 of the glove 70 before or during a patient monitoring session. The pocket 72 (or the opening for the pocket 72) may be disposed on an exterior surface of the glove 70, so that the pocket 72 may be easily accessed when the glove 70 is applied to the patient. In other embodiments, the pocket 72 may be disposed on an interior surface of the glove 70 that is adjacent to the patient's skin when the glove 70 is applied to the patient, thus providing additional protection and securement of the reusable portion 16. In such cases, the reusable portion 16 may be replaced by removing the reusable portion 16 from the pocket 72 and inserting a new (e.g., charged) reusable portion 16 into the pocket 72. The new reusable portion 16 may then be connected to the disposable portion 14 to provide power to the disposable portion 14 during longer monitoring sessions, for example. In some embodiments, one or more pockets 72 may be provided in the glove 70 to hold multiple reusable portions 16, the disposable portion 14, the flex circuit 18, and/or other components of the sensor 12.

In certain embodiments, the glove 70 may be disposable and the disposable portion 14 and/or the flex circuit 18 (or a cable or a wire) may be integrated into or attached to the glove 70. More specifically, in certain embodiments, the disposable portion 14 and/or the flex circuit 18 may be sewn or woven into the glove 70. Thus, in operation, the disposable portion 14 and/or the flex circuit 18 may be applied to the patient when the glove 70 is placed on the hand of the patient. The reusable portion 16 may then be electrically coupled to the disposable portion 14 via the flex circuit 18, for example, and patient monitoring may commence. At the conclusion of a monitoring session, the disposable portion 14 and/or the flex circuit 18 may be disconnected from the reusable portion 16. The disposable glove 70, along with the integrated disposable portion 14 and/or the integrated flex circuit 18, may be discarded.

In alternate embodiments, the reusable portion 16 may be attached to or integrated into the glove 70. In such cases, the glove 70 may not be disposable, but may be cleaned and disinfected for use with different patients and/or may be reused in multiple different monitoring sessions for the same patient. Thus, the glove 70 and the reusable portion 16 may form a reusable unit and may be connected to a disposable portion 14 (e.g., via the flex circuit 18) for monitoring the patient. At the conclusion of the monitoring session, the glove 70 and the reusable portion 16 may be reused for the same patient at a later time, transferred from one patient to another, or may be reused with various disposable portions 16, for example.

Additionally, the glove 70 may be a fingerless glove (e.g., the glove 70 does not cover the fingertips of the patient) as shown in FIG. 5. Such a configuration may allow a medical practitioner to access the fingertips of the patient to conduct various physiological tests and assess the patient's condition. In certain embodiments, the glove 70 may extend over one or more fingertips of the patient so as to cover, protect, and/or secure the disposable portion 14 of the sensor 12.

As noted above, placing the reusable portion 16 away from the measurement site, such as on the back of the hand of the patient, may be more comfortable for the patient in certain cases and may accommodate large components, such as a large battery and/or large processing components. However, in some circumstances, it may be desirable to position the disposable portion 14 and the reusable portion 16 in close physical proximity. For example, placing the disposable portion 14 and the reusable portion 16 in proximity to each other may reduce noise within the system 10. Additionally, in some embodiments, it may be desirable to position the disposable portion 14 and the reusable portion 16 closely enough so that the detector 44 may be incorporated into the reusable portion 16 of the sensor 12. The detector 44 is often a relatively expensive component of the sensor 12, and thus, incorporating the detector 44 into the reusable portion 16 of the sensor 12 may provide significant cost savings. Additionally, disposing the detector 44 within the reusable portion 16 may enable the disposable portion 14 to be smaller, thus resulting in additional cost savings. Moreover, relatively less shielding, or in some cases no shielding, may be required when the detector 44 is disposed within the reusable portion 16 (or in a different portion than the emitter 44), leading to additional cost savings, as well as a less complex design. More particularly, various shielding components are typically employed in sensors 12 to isolate the detector 44 from noise interference. In the present embodiments, shielding may be provided proximate to the detector 44 and/or to the circuitry 62. However, where the emitter 42 is in the disposable portion 14 and the detector 44 is within the reusable portion 16, and the portions 14, 16 are coupled via a wire or a cable, shielding may not be required at or proximate to the detector 44. Furthermore, in embodiments where the portions 14, 16 are joined by a flexible connection, such as a flex circuit, the sensor 12 may be configured to emit light and detect light without any shielding means. In other words, the sensor 12 may be configured to enable the detector 44 to accurately detect the reflected or transmitted light for patient monitoring (i.e., with no interference, or with an acceptable amount or level of interference).

Accordingly, FIG. 6 illustrates an embodiment of the sensor 12 having the emitter 42 within the disposable portion 14 and the detector 44 within the reusable portion 16. FIG. 7 and FIG. 8 depict a cross-sectional side view and a bottom view, respectively, of the reusable portion 16 having the detector 44 disposed therein. Although certain embodiments and arrangements of sensor components are depicted in FIGS. 6-8, it should be understood that the other sensor components (e.g., the calibration element 46, the battery 60, the wireless module 32, and/or the battery meter 80) may be disposed or distributed in the disposable portion 14 and/or the reusable portion 16 in any suitable manner. For example, although the battery 60 and the battery meter 80 are depicted and described as positioned within the reusable portion 16, it is envisioned that the battery 60 and/or the battery meter 80 may be disposable, and one or both of these components may be included in the disposable portion 14.

As mentioned above, the portions 14, 16 of the sensor 12 may be configured to be disposed on a variety of tissue locations on the patient, such as on a finger, a toe, a foot, an ankle, a wrist, or a forehead. The particular embodiments of the sensor 12 of FIGS. 6-8 may be well-suited for positioning on the patient's forehead, although the sensor 12 may also be positioned in other appropriate locations, such as the patient's abdomen, back, foot, or the like. Additionally, in certain cases, the sensor 12 may be configured to be disposed on an ankle or a wrist of a neonate for monitoring, as described in more detail below. Regardless of the intended location for the sensor 12, various elements may be utilized to secure and/or couple the sensor 12 to the patient, as described in more detail below.

FIG. 6 illustrates an embodiment of the sensor 12 having the detector 44 disposed within the reusable portion 16. Additionally, the reusable portion 16 may include the battery 60, the wireless module 32, and/or the circuit board 62 disposed within the body 58. In some embodiments, a battery meter 80 may be provided within the reusable portion 16. The battery meter 80 may provide a visual indication of the remaining battery life, for example. In certain embodiments, the detector 44 may be incorporated or integrated into the circuit board 62. As shown, the disposable portion 14 of the sensor 12 may include the emitter 42, and in some embodiments, the calibration element 46. The detector 44 may be configured to detect light that is emitted by the emitter 42. Additionally, the battery 60 may power components within the disposable portion 14, such as the emitter 42. Furthermore, the circuit board 62 may control operation of the emitter 42 within the disposable portion 14. Thus, the components within the disposable portion 14 and the reusable portion 16 work together for patient monitoring.

The disposable portion 14 and the reusable portion 16 of the sensor 12 depicted in FIG. 6 may be coupled together through any suitable means, such as via the connector 48, as described above with respect to FIG. 2. In other embodiments, the disposable portion 14 and the reusable portion 16 may be coupled together via the flex circuit 18, as described above with respect to FIG. 3. Furthermore, in certain embodiments, the disposable portion 14 and the reusable portion 16 may include contacts 82 that, when joined, electrically connect the disposable portion 14 and the reusable portion 16. Thus, the disposable portion 14 and the reusable portion 16 may be coupled together or snapped together by bringing the contacts 82 towards each other as shown by arrows 84. When joined in such a way, the connection between the disposable portion 14 and the reusable portion may be flexible (e.g., a flex connection), thus allowing movement or bending about the connection. In some embodiments, the sensor bodies 40, 58 are not in contact and/or do not overlap, except at the contacts 82 or the ends of the sensor bodies 40, 58 supporting the contacts 82. In other words, the portions 14, 16 are placed generally adjacent with respect to one another, coupled together only by contacts 82. Additionally, in some embodiments, the detector 44 may be isolated from noise interference by shielding disposed proximate to the detector and/or the signal processing hardware within the reusable portion 16. As noted above, in some embodiments, the disposable portion 14 and the reusable portion 16 may be flexibly coupled to on another, and the reusable portion 16 may be configured to enable the detector 44 to accurately and/or suitably detect the reflected or transmitted light without any shielding means disposed about the detector 44 and/or without any shielding means disposed within the reusable portion 16.

FIG. 7 illustrates a side view and FIG. 8 illustrates a bottom view of the reusable portion 16 of the sensor 12 of FIG. 6. As shown in FIG. 7, the reusable portion 16 may have a curvature to generally correspond to the curvature of the forehead of the patient. Additionally, as shown in both FIG. 7 and FIG. 8, a window 88 may be disposed along the bottom surface 86 of the reusable portion 16 to enable the reflected or transmitted light from the tissue of the patient to reach the detector 44. The window 88 may be formed from any suitable material that is substantially transparent with respect to the wavelengths of light used by the emitter 42. For example, the window 88 may be formed from any of a variety of suitable elastomeric compositions, including acrylic elastomers, polyimide, silicones, silicone rubber, celluloid, PMDS elastomer, polyurethane, polypropylene, polyethylene, acrylics, nitrile, PVC films, acetates, and latex.

FIGS. 9-11 depict a band 90 (e.g., a headband) that may be utilized to couple the sensor 12 to the tissue of the patient. Although the discussion below describes placement of the sensor 12 and the headband 90 on the forehead of the patient, it should be understood that the sensor 12 and/or the headband 90 described herein may be readily adapted to be disposed on various tissue locations on the patient to facilitate patient monitoring.

In particular, FIG. 9 illustrates the headband 90 that may be configured to wrap around the head of the patient. The headband 90 may include an adjustment mechanism 91 that enables the headband 90 to adjust and change size (e.g., circumference) to adapt to the different anatomies of various patients. In some embodiments, the headband 90 may include an elastic material that enables the headband 90 to adjust in size and to fit securely to the patient. The headband 90 may also be disposable or reusable, as described further below. In certain embodiments, the headband 90 may be configured to be positioned over the sensor 12. Thus, the disposable portion 14 and the reusable portion 16 of the sensor 12 may be connected (via the connector 48 or the flex circuit 18, for example) and applied to the tissue of the patient. The headband 90 may then be wrapped over or placed over the sensor 12, protecting the sensor 12 and applying a normal force against the sensor 12 to secure the sensor 12 to the forehead of the patient for patient monitoring. In such embodiments, the headband 90, or a portion of the headband 90 that is configured to be placed over the sensor 12 or the reusable portion 16 of the sensor 12, may be transparent to enable the operator to visualize the battery meter 80.

However, in some embodiments, as shown in FIGS. 9 and 10, the sensor 12 may be applied over the headband 90 (e.g., the sensor 12 may be mounted on the headband 90). In such cases, the headband 90 may have a top surface 92 and a patient contacting surface 94. The headband 90 may also have a mounting element or surface 96 for mounting the portions 14, 16 of the sensor 12 to the headband 90. All or some of the mounting surface 96 may be adhesive and/or transparent to wavelengths of light emitted by the emitter 42. Additionally or alternatively, other features such as snaps, clips, or hook and loop fabric may be utilized to couple one or both of the portions 14, 16 of the sensor 12 to the headband 90. In certain embodiments, the mounting surface 96 may be disposed over or overlap with a transparent window 98 that extends between the patient contacting surface 94 and the mounting surface 96. Specifically, the transparent window 98 may facilitate the transmission of light emitted by the emitter 42 through the headband 90 and into the patient's tissue, as well as detection of the reflected (or transmitted) light by the detector 44. Thus, the portions 14, 16 of the sensor 12 may be mounted on the mounting surface 96 such that the emitter 42 and detector 44 are each positioned over the transparent portion of the mounting surface 96 and over the transparent windows 98. The emitter 42 may emit light through the transparent window 98, while the detector 44 may receive the reflected light through the transparent window 98. In some embodiments, both the mounting surface 96 and the transparent window 98 may generally have a size, shape, and configuration that correspond to the sensor 12. In yet other embodiments, one or more transparent windows 98 may be provided to correspond with the size and/or location of the emitter 42 and detector 44 (e.g., one or more transparent windows 98 are aligned with the emitter 42 and the detector 44) to facilitate transmission of light through the headband 90 for patient monitoring. Furthermore, in some embodiments, as shown in FIG. 10, the mounting surface 96 may be recessed within the headband 90 (e.g., the transparent window 98 may be thinner than other portions of the headband 90). In such cases, the sensor 12 may generally fit within a recess 100 and the sensor 12 may be surrounded by and protected by portions of the headband 90, while also being accessible for replacement of the disposable portion 14 and/or the reusable portion 16, for example. Additionally, mounting the sensor 12 on the mounting surface 96 of the headband 90 also enables visualization of the battery meter 80. In some embodiments, rather than the mounting surface 96 and/or the transparent window 98, the headband 90 may include an aperture or a hole configured to allow light to pass between the sensor 12 and the patient.

In certain embodiments, as shown in FIG. 11, the headband 90 may include one or more pockets 102 configured to receive and to hold the disposable portion 14 and/or the reusable portion 16. The one or more pockets 102 may have a bottom layer 104 and a sensor securing layer 106. At least a portion of the bottom layer 104 of the pocket 102 may be transparent to the wavelengths of light emitted by the emitter 42. Thus, the disposable portion 14 and/or the reusable portion 16 may fit within the pocket 102, and the optical components (e.g., the emitter 42 and detector 44) may emit and detect light through the transparent portion of the bottom layer 104. The bottom layer 104 may also be disposed over or overlap with the transparent window 98, which extends between the bottom layer 104 and the patient contacting layer 94 of the headband 90 to facilitate light transmission between the sensor 12 within the pocket 102 and the patient. In some cases, the transparent window 98 may form the bottom layer 104 of the pocket 102. In certain embodiments, the bottom layer 104 and/or the securing layer 106 may have an adhesive material and/or a gripping material (e.g., a material with a high coefficient of friction) that adheres to or grips the disposable portion 14 and/or the reusable portion 16, thus coupling the portions 14, 16 to the headband 90 and securing the portions 14, 16 within the pocket 102. The pocket 102 may be configured to be easily opened for removal or replacement of the disposable portion 14 and/or reusable portion 16. Additionally, the securing layer 106 may be transparent to enable the operator to visualize the sensor 12 within the pocket 102, or the securing layer 106 may have a transparent portion configured to be positioned over the battery meter 90 to enable the operator to visualize the battery meter 80 during patient monitoring, for example.

In some embodiments, the disposable portion 14 may be attached to or integrated into the headband 90. More specifically, the disposable portion 14 may be sewn or woven into the headband 90, and the disposable portion 14 may be applied to the tissue of the patient when the headband 90 is placed on the patient. The disposable portion 14 may be integrated into the headband 90 such that the disposable portion 14 directly contacts the patient's skin when the headband 90 is placed on the patient. However, in other embodiments, the disposable portion 14 may be integrated into the headband 90 adjacent to (e.g., over) the transparent window 98 of the headband 90, such that the emitted light may pass from the emitter 42 and through the transparent window 98 into the patient's tissue. In embodiments where the disposable portion 14 is integrated into the headband 90, the headband 90 may also include a mounting surface 96 for receiving and/or mounting the reusable portion 16. Thus, the reusable portion 16 may be mounted on the mounting surface 96 and coupled to the integrated disposable portion 14 via the flex circuit 18 or via contacts 82, for example. In some embodiments, the headband 90 may include the pocket 102 for receiving and holding the reusable portion 16. Thus, the reusable portion 16 may be placed within the pocket 102 and coupled to the integrated disposable portion 14 for patient monitoring. At the conclusion of a monitoring session, the reusable portion 16 may be disconnected from the disposable portion 14 and removed from the pocket 102, and the headband 90 and the integrated disposable portion 14 may be discarded as a unit.

In other embodiments, the reusable portion 16 may be integrated into the headband 90. In such cases, the headband 90 may not be disposable, but may be cleaned and disinfected for use with different patients and/or reused in multiple different monitoring sessions for the same patient. Thus, at the conclusion of a monitoring session, the disposable portion 14 may be removed and discarded, while the headband 90 and the reusable portion 16 may be removed as a unit and/or reused with various disposable portions 14.

In certain circumstances, such as in monitoring neonates, it may be desirable to position the sensor 12 on the patient's wrist or ankle. While the sensor 12 may be similar for adult and neonatal applications, in some embodiments, the sensor 12 may have a different configuration for neonates. One such possible configuration is shown in FIG. 12. As depicted, the disposable portion 14 may be generally elongated and/or may be configured so that the emitter 42 is disposed relatively far from the contacts 82 (e.g., the emitter 42 is disposed near a first end 112 of the disposable portion 14, while the contacts 82 are disposed near a second end 110 of the disposable portion 14). Such elongation of the disposable portion 14 and/or placement of the emitter 42 may provide the proper distance between the emitter 42 of the disposable portion 14 and the detector 44 of the reusable portion 16 when the portions 14, 16 are coupled together and placed on the neonate's wrist or ankle for monitoring. As described above, the disposable portion 14 and the reusable portion 16 of the sensor 12 may be coupled together via the flex circuit 18 or via the contacts 82, for example. The disposable portion 14 may include various sensor components, such as the emitter 42, and in some cases, the calibration element 46. The reusable portion 16 may include various components, such as the detector 44, the wireless module 32, the battery 60, the circuitry 62, and/or the battery meter 80, for example.

In neonatal application, it may be desirable to place the sensor 12 on or near the patient's wrist or ankle for patient monitoring. Thus, in some embodiments, a band 120 or a wrap 130 configured to protect the sensor 12 and to couple the sensor 12 to the wrist or ankle of the patient is provided. The band 120 or the wrap 130 configured to wrap around the patient's wrist or ankle may also beneficially secure the sensor 12 to the patient without the need for adhesives, which may injure or irritate the fragile skin of neonates. FIGS. 13-15 depict embodiments of the band 120 and the wrap 130 that are configured to couple the sensor 12 to the wrist or the ankle of the patient. Specifically, FIG. 13 depicts an embodiment of the band 120 and FIG. 14 depicts an embodiment of the wrap 130. Additionally, FIG. 15 depicts a side cross-sectional view of an embodiment of the sensor 12 mounted on the wrap 130. The band 120 and the wrap 130 may take any of a variety of suitable configurations, and may be either disposable or reusable. The band 120 and the wrap 130 may be formed from an elastic material or combination of part rigid and part elastic material that enables the band 120 and the wrap 130 to stretch and/or to adjust in size and to adapt to different patient anatomies.

In certain embodiments, the band 120 may generally be configured to slide over and to circumferentially surround the ankle or the wrist of the neonate patient. In contrast, the wrap 130 may include extensions 132 that partially or completely wrap around the wrist or ankle of the patient. In certain embodiments, the wrap 130 may include a first extension 132a and a second extension 132b that are configured to wrap around the wrist or the ankle and to attach to each other. The extensions 132a, 132b may attach to each other via any suitable mechanism, such as via a hook and loop fabric, for example. Although embodiments of the band 120 and the wrap 130 have some structural differences (e.g., extensions 132), embodiments of the band 120 and the wrap 130 may also share certain features to secure the sensor 12 to the wrist or ankle of the patient, and thus are described together below.

As with the headband 90, the band 120 and the wrap 130 may take any of a variety of configurations to couple the sensor 12 to the patient. For example, the band 120 and the wrap 130 may be configured to be wrapped over or positioned over the sensor 12. Thus, the disposable portion 14 and the reusable portion 16 of the sensor 12 may be coupled together and applied to the patient's skin. The band 120 or the wrap 130 may then be wrapped over or placed over the sensor 12, protecting the sensor 12 and applying a normal force against the sensor 12 to secure the sensor 12 to the wrist or ankle of the patient for monitoring. In such cases, the band 120 or the wrap 130 may be transparent or may include a transparent portion to enable visualization of the battery meter 80.

In other embodiments, as shown in FIGS. 13-15, the sensor 12 may be applied over the band 120 or the wrap 130 (e.g., the sensor 12 may be mounted on the band 120 or the wrap 130). In such cases, the band 120 or the wrap 130 may have a patient contacting surface 134 and a top surface 136, as best shown in FIG. 15. The band 120 or the wrap 130 may also have a mounting surface 138 for receiving and mounting the portions 14, 16 of the sensor 12 on the band 120 or to the wrap 130. In some embodiments, the mounting surface 138 may include an adhesive to facilitate coupling of the portions 14, 16 to the band 120 or the wrap 130. Although not shown, the portions 14, 16 may be mounted to the band 120 or the wrap 130 through various mechanical elements, such as snaps or clips, for example.

In some embodiments, a single mounting surface 138 having a size and shape that generally corresponds to the sensor 12 may be provided to mount the sensor 12 onto the band 120 or the wrap 130. However, in other embodiments, as shown in FIGS. 13-15, a first mounting surface 138a may be provided for the disposable portion 14, while a second mounting surface 138b may be provided for the reusable portion 14. In some embodiments, at least a portion of the mounting surface 138 may be transparent to wavelengths of light emitted by the emitter 42. In certain embodiments, the mounting surface 138 may be disposed over or overlap with a transparent window 140. The transparent window 140 may extend between the patient contacting surface 134 and the top surface 136, and may generally align with the mounting surface 138, or with a portion of the mounting surface 138. In some embodiments, the portions 14, 16 are mounted directly onto the transparent window 140 in lieu of the mounting surface 138 (in other words, the transparent window 140 may have an adhesive surface and may form the mounting surface 138).

The transparent window 140 may be generally configured to facilitate transmission of the light emitted by the emitter 42 through the band 120 or the wrap 130 and into the patient's tissue. The transparent window 140 may also be configured to enable detection of the reflected (or transmitted) light by the detector 44. Thus, in some embodiments, the portions 14, 16 of the sensor 12 may be mounted on the mounting surface 138 and such that the emitter 42 and the detector 44 are each positioned over (e.g., adjacent) the transparent portion of the mounting surface 138 and over the transparent window 140. In some embodiments, both the mounting surface 138 and the transparent window 140 may generally have a size, shape, and configuration that correspond to the sensor 12. However, in other embodiments, one or more transparent windows 140 may be provided to correspond with and align with the emitter 42 and detector 44 when the portions 14, 16 are coupled to the band 120 or the wrap 130. For example, as shown in FIGS. 13-15, a first transparent window 140a is provided to align with the emitter 42 of the disposable portion 14, and a second transparent window 140b is provided to align with the detector 44 of the reusable portion 16. As shown in FIG. 13, the first transparent window 140a and the second window 140b may be spaced apart at a certain distance 126, corresponding to the distance between the emitter 42 and the detector 44 when the portions 14, 16 of the sensor 12 are coupled together. In some embodiments, the distance 126 between the first transparent window 140a (and/or the emitter 42) and the second transparent window 140b (and/or the detector 44) may be between about 5 to about 50 millimeters (mm). In some embodiments, the distance 126 may be between about 10 to about 40, about 15 to about 30, or about 20 to about 25 mm.

Additionally, alignment indicia may be provided, such as visible lines or marks on the band 120 or the wrap 130 to enable the operator to visually confirm proper placement of the portions 14, 16 with respect to the mounting surface 138 and/or the transparent windows 140. Furthermore, in some embodiments, the mounting surface 138 and/or the transparent window 140 may be recessed so that the sensor 12 may generally fit within the recess of the band 120 or the wrap 130, as best shown in FIG. 15. In such configurations, the sensor 12 may be surrounded by and protected by portions of the band 120 or the wrap 130, while also being easily accessible for replacement of the disposable portion 14 or the reusable portion 16, for example. In certain embodiments, rather than the mounting surface 138 and/or the transparent window 140, the band 130 or the wrap 140 may provide an opening or an aperture extending between the patient contacting surface 134 and the top surface 136. In such cases, the aperture may be configured to facilitate the transmission of light from the emitter 42 to the patient's tissue and to the detector 44 from the patient's tissue for patient monitoring.

In some embodiments, it may be desirable to provide a protective layer or securing layer to cover the sensor 12 when the sensor 12 is coupled to the band 120 or the wrap 130. Such securing layers may be particularly useful in neonatal applications, where sensors 12 are likely to be dislodged by patient movement, for example. Thus, in some embodiments, the band 120 or the wrap 130 may include one or more securing layers configured to be placed over the portions 14, 16 of the sensor 12 when mounted to the band 120 or the wrap 130. The securing layer combined with other portions of the band 120 or the wrap 130 (e.g., the top layer 136 and/or the mounting surface 138 and/or the transparent window 140) may generally form a pocket to receive and to hold the disposable portion 14 and/or the reusable portion 16. The pocket formed on the band 120 or the wrap 130 may be similar to the pocket 102 described above with respect to FIG. 11. When the disposable portion 14 and/or the reusable portion 16 are placed within the pocket, the optical components (e.g., the emitter 42 and detector 44) may emit and detect light through the band 120 or the wrap 130 via the transparent window 140, for example. In certain embodiments, the securing layer may have an adhesive material or a gripping material that adheres to or grip the disposable portion 14 and/or the reusable portion 16, thus coupling the portions 14, 16 to the band 120 or the wrap 130 and securing the portions 14, 16 within the pocket. Where a securing layer is provided, the securing layer may be transparent to enable visualization of the sensor 12 within the pocket, or a portion of the securing layer may be transparent to align with and enable visualization of the battery meter 80.

In some embodiments, the disposable portion 14 may be attached to or integrated into the band 120 or the wrap 130. More specifically, the disposable portion 14 may be sewn or woven into the band 120 or the wrap 130, and the disposable portion 14 may be applied to the tissue of the patient when the band 120 or the wrap 130 is placed on the patient. In such cases, the disposable portion 14 may be positioned so that the disposable portion 14 directly contacts the patient's skin when the band 120 or the wrap 130 is placed on the patient. However, in other embodiments, the disposable portion 14 may be integrated into the band 120 or the wrap 130 adjacent to (e.g., over) a transparent window 140, such that the emitted light may pass from the emitter 42 and through the transparent window 140 into the patient's tissue. In embodiments where the disposable portion 14 is integrated into the band 120 or the wrap 130, the band 120 or the wrap 130 may include a mounting surface 138 for receiving and/or mounting the reusable portion 16. Thus, the reusable portion 16 may be mounted on the mounting surface 138 and coupled to the integrated disposable portion 14 via the flex circuit 18 or via contacts 82, for example. In some embodiments, the band 120 or the wrap 130 may include the securing layer that forms the pocket for receiving and holding the reusable portion 16. Thus, the reusable portion 16 may be placed within the pocket and coupled to the integrated disposable portion 14 for patient monitoring. At the conclusion of a monitoring session, the reusable portion 16 may be disconnected from the disposable portion 14 and removed from the pocket, while the integrated disposable portion 14 and the band 120 or the wrap 130 may be discarded as a unit.

In other embodiments, the reusable portion 16 may be integrated into the band 120 or the wrap 130. In such cases, the band 120 or the wrap 130 may not be disposable, but may be cleaned and disinfected for use with different patients and/or reused in multiple different monitoring sessions for the same patient. Thus, at the conclusion of a monitoring session, the disposable portion 14 may be removed and discarded, while the band 120 or the wrap 130 and the integrated reusable portion 16 may be either remain on the patient for use with a second disposable portion 14 or may be removed as a unit and reused for various patients.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, individual features of the disclosed embodiments may be combined or exchanged.

What is claimed is:

1. A wireless medical sensor comprising:
a disposable portion comprising an emitter; and
a reusable portion configured to be removably coupled to the disposable portion, wherein the reusable portion comprises a battery configured to provide power to the emitter and a wireless module configured to communicate with a monitor.

2. The medical sensor of claim 1, wherein the disposable portion comprises a detector configured to detect light emitted by the emitter after the light passes through a tissue of a patient.

3. The medical sensor of claim 1, wherein the reusable portion comprises a detector configured to detect light emitted by the emitter after the light passes through a tissue of a patient.

4. The medical sensor of claim 1, wherein the disposable portion comprises a calibration element and the wireless module is configured to provide information stored within the calibration element to the monitor.

5. The medical sensor of claim 1, wherein the disposable portion and the reusable portion are coupled together via a flex circuit.

6. The medical sensor of claim 1, wherein the reusable portion has an annular shape and is configured to fit around a digit of a patient.

7. The medical sensor of claim 1, wherein the reusable portion is configured to be coupled to a glove.

8. The medical sensor of claim 7, wherein the disposable portion and the reusable portion are configured to be coupled via a wire that is integrated into the glove.

9. The medical sensor of claim 1, wherein the disposable portion and the reusable portion are configured to be mounted onto a mounting surface of a band that is configured to be applied to a patient.

10. A medical monitoring system comprising:
a monitor; and
a wireless medical sensor comprising:
a disposable portion comprising an emitter; and
a reusable portion configured to be removably coupled to the disposable portion, wherein the reusable portion comprises a battery configured to provide power to the emitter and a wireless transceiver configured to communicate with the monitor.

11. The medical monitoring system of claim 10, wherein the disposable portion comprises a calibration element.

12. The medical monitoring system of claim 11, wherein the wireless transceiver is configured to provide information stored in the calibration element to the monitor.

13. The medical monitoring system of claim 10, wherein the reusable portion comprises a detector configured to detect light after the light passes through a tissue of a patient.

14. The medical monitoring system of claim 10, wherein monitor is configured to wirelessly provide instructions to the wireless medical sensor.

15. The medical monitoring system of claim 10, wherein the disposable portion and the reusable portion are coupled via a flex circuit.

16. The medical monitoring system of claim 10, comprising a band configured to be applied to a patient, wherein the disposable portion is integrated into the band.

17. The medical monitoring system of claim 16, wherein the reusable portion is configured to be mounted on a mounting surface of the band.

18. A medical sensor system comprising:
a band configured to be applied to a tissue of a patient;
a wireless medical sensor comprising:
a disposable portion comprising an emitter, the disposable portion being integrated into the band; and
a reusable portion configured to be removably coupled to the disposable portion, the reusable portion comprising a battery configured to provide power to the emitter and a wireless transceiver configured to communicate with a monitor.

19. The medical sensor system of claim 18, wherein the reusable portion comprises a detector configured to detect light after the light passes through a tissue of a patient.

20. The medical sensor system of claim 18, wherein the disposable portion comprises a calibration element.
